(19) United States
(12) Patent Application Publication
(10) Pub. No.: US 2012/0316442 A1
(43) Pub. Date: Dec. 13, 2012

(54) Hypothesis Validation of Far Wall Brightness in Arterial Ultrasound

(75) Inventor: Jasjit S. Suri, Roseville, CA (US)

(73) Assignee: AtheroPoint LLC, Roseville, CA (US)

(21) Appl. No.: 13/589,802

(22) Filed: Aug. 20, 2012

Related U.S. Application Data
(63) Continuation-in-part of application No. 12/798,424, filed on Apr. 2, 2010.
Application No. 12/798,424 is a continuation-in-part of application No. 12/799,177, filed on Apr. 20, 2010.
Application No. 12/799,177 is a continuation-in-part of application No. 12/799,558, filed on Apr. 26, 2010.
Application No. 12/799,558 is a continuation-in-part of application No. 12/802,431, filed on Jun. 7, 2010.
Application No. 12/802,431 is a continuation-in-part of application No. 12/896,875, filed on Oct. 2, 2010.
Application No. 12/896,875 is a continuation-in-part of application No. 12/960,491, filed on Dec. 4, 2010.
Application No. 12/960,491 is a continuation-in-part of application No. 13/053,971, filed on Mar. 22, 2011.
Application No. 13/053,971 is a continuation-in-part of application No. 13/077,631, filed on Mar. 31, 2011.
Application No. 13/077,631 is a continuation-in-part of application No. 13/107,935, filed on May 15, 2011.
Application No. 13/107,935 is a continuation-in-part of application No. 13/219,695, filed on Aug. 28, 2011.
Application No. 13/219,695 is a continuation-in-part of application No. 13/253,952, filed on Oct. 5, 2011.
Application No. 13/253,952 is a continuation-in-part of application No. 13/407,602, filed on Feb. 28, 2012.
Application No. 13/407,602 is a continuation-in-part of application No. 13/412,118, filed on Mar. 5, 2012.
Application No. 13/412,118 is a continuation-in-part of application No. 13/449,518, filed on Apr. 18, 2012.
Application No. 13/449,518 is a continuation-in-part of application No. 13/465,091, filed on May 7, 2012.
(60) Provisional application No. 61/525,745, filed on Aug. 20, 2011.

(51) Int. Cl. A61B 8/13

(52) U.S. Cl. 600/443

(57) ABSTRACT

Automated IMT system hypothesize that far wall of the common carotid artery has the highest intensity. In this current application, we verify that this hypothesis holds true for B-mode or RF-mode longitudinal ultrasound images of the carotid wall. The methodology consists of generating the composite image (arithmetic sum of images) from the database by first registering the carotid image frames with respect to a nearly straight carotid artery frame from the same database using (a) B-spline based non-rigid registration and (b) affine registration. Prior to registration, we segment the carotid artery lumen using a level set based algorithm followed by morphological image processing. The binary lumen images are registered and the transformations are applied to the original grayscale CCA images. These B-mode or RF-mode ultrasound images are then used for IMT computation using automated methods which hypothesize that far wall has the brightest intensity distribution.

### PRIORITY PATENT APPLICATIONS

This is a continuation-in-part patent application of co-pending patent application Ser. No. 12/798,424; filed Apr. 2, 2010 by the same applicant. This is also a continuation-in-part patent application of co-pending patent application Ser. No. 12/799,177; filed Apr. 20, 2010 by the same applicant. This is also a continuation-in-part patent application of co-pending patent application. Ser. No. 12/799,558; filed Apr. 26, 2010 by the same applicant. This is also a continuation-in-part patent application of co-pending patent application Ser. No. 12/802,431; filed Jun. 7, 2010 by the same applicant. This is also a continuation-in-part patent application of co-pending patent application Ser. No. 12/896,875; filed Oct. 2, 2010 by the same applicant. This is also a continuation-in-part patent application of co-pending patent application Ser. No. 12/960,491; filed Dec. 4, 2010 by the same applicant. This is also a continuation-in-part patent application of co-pending patent application Ser. No. 13/053,971; filed Mar. 22, 2011 by the same applicant. This is also a continuation-in-part patent application of co-pending patent application Ser. No. 13/077,631; filed Mar. 31, 2011 by the same applicant. This is also a continuation-in-part patent application of co-pending patent application Ser. No. 13/107,935; filed May 15, 2011 by the same applicant. This is also a continuation-in-part patent application of co-pending patent application Ser. No. 13/219,695; filed Aug. 28, 2011 by the same applicant. This is also a continuation-in-part patent application of co-pending patent application Ser. No. 13/253,952; filed Oct. 5, 2011 by the same applicant. This is also a continuation-in-part patent application of co-pending patent application Ser. No. 13/407,602; filed Feb. 28, 2012 by the same applicant. This is also a continuation-in-part patent application of co-pending patent application Ser. No. 13/412,118; filed Mar. 5, 2012 by the same applicant. This is also a continuation-in-part patent application of co-pending patent application Ser. No. 13/449,518; filed Apr. 18, 2012 by the same applicant. This is also a continuation-in-part patent application of co-pending patent application Ser. No. 13/465,091; filed May 7, 2012 by the same applicant. This present patent application claims priority to the referenced co-pending patent applications.

This present non-provisional patent application also claims priority to U.S. provisional patent application Ser. No. 61/525,745; filed Aug. 20, 2011 by the same applicant. The entire disclosures of the referenced co-pending patent applications and the provisional patent application are considered part of the disclosure of the present application and are hereby incorporated by reference herein in their entirety.

### TECHNICAL FIELD

This patent application relates to methods and systems for use with data processing, validation of imaging systems, according to one embodiment, and more specifically, for ultrasound image processing.

### BACKGROUND)

Atherosclerosis is the thickening and narrowing of the arteries due to formation of plaque on the walls of the artery. It is one of the leading causes of stroke and is the first clinical manifestation of cardiovascular disease. Recent research has been focused on determining early indicators of atherosclerosis. IMT is an early indicator of atherosclerosis and precedes luminal narrowing due to plaque formation. Since plaque formation starts in the walls of the artery, IMT could be a better indicator than lumen area or blood velocity. Population studies have shown a strong correlation between carotid IMT and several cardiovascular risk factors and IMT has also been found to be associated with the extent of atherosclerosis and end organ damage of high-risk patients. B-mode ultrasound (US) or RF-mode ultrasound is a non-invasive method to measure IMT especially in easily accessible arteries like the carotid. IMT measurements using ultrasonography correlate well with histopathology and are reproducible.

The state of Atherosclerosis in carotids or other blood vessels can be studied using magnetic resonance imaging (MRI) or Ultrasound imaging. Because ultrasound offers several advantages like real time scanning of blood vessels, compact in size, low cost, easy to transport (portability), easy availability and visualization of the arteries are possible, Atherosclerosis quantification is taking a new dimension using ultrasound. Because one can achieve compound and harmonic imaging, which generates high quality images with ultrasound, it is thus possible to do two-dimensional (2D) and three-dimensional (3D) imaging of blood vessel ultrasound images for monitoring of Atherosclerosis.

In recent years, the possibility has arisen of adopting a composite thickness of the tunica intima and media, an intima-media thickness (hereinafter referred to as an “IMT” or “CIMT”) of carotid arteries, as surrogate marker for cardiovascular risk and stroke. Conventional methods of imaging a carotid artery using an ultrasound system, and measuring the IMT using an ultrasonic image for the purpose of diagnosis are being developed.

A conventional measuring apparatus can measure an intima-media thickness of a blood vessel using an ultrasound device to scan the blood vessel. Then, for example, an image of a section of the blood vessel including sections of the intima, media and adventitia is obtained. The ultrasound device further produces digital image data representing this image, and outputs the digital image data to a data analyzing device.

The intima, media and adventitia can be discriminated on the basis of changes in density of tissue thereof. A change in density of tissue of the blood vessel appears as a change of luminance values in the digital image data. The data analyzing device detects and calculates the intima-media thickness on the basis of the changes of luminance values in the digital image data. The digital image data can include a plurality of luminance values each corresponding to respective one of a plurality of pixels of the image. The data analyzing device can set a base position between a center of the blood vessel and a position in a vicinity of an inner intimal wall of the blood vessel on the image, on the basis of a moving average of the luminance values. The data analyzing device can detect a maximum value and a minimum value from among the luminance values respectively corresponding to a predetermined number of the pixels arranged from the base position toward a position of an outer adventitial wall on the image. The data analyzing device can then calculate the intima-media thickness on the basis of the maximum value and the minimum value.

The major challenges which can be affected in finding the IMT are: (a) how well the ultrasound probe is gripped with the neck of a patient to scan the carotids; (b) how well the ultrasound gel is being applied; (c) the orientation of the probe; (d) demographics of the patient; (e) presence of calcium in the proximal walls; (f) skills of the sonographer or vascular surgeon; and (g) the threshold chosen for finding the peaks corresponding to the lumen-intima (LI) border points, and the media-adventitia (MA) border points (collectively denoted herein as the LIMA or LIMA points) for each signal orthogonal to the lumen. These challenges have complicated IMT measurement using conventional systems.

### BRIEF DESCRIPTION OF THE DRAWINGS

The various embodiments are illustrated by way of example, and not by way of limitation, in the figures of the accompanying drawings in which:

FIG. 1 shows an example of ultrasound image of a blood vessel, particularly the Carotid Artery. FIG. 1 shows the near and far wall of the common carotid artery.

FIG. 2 shows system for validation of highest brightness of the far wall of the blood vessel, particular common carotid artery.

FIG. 3 the expanded version of the validation processor.

FIG. 4 shows the processor for computation of Alignment Parameters for the database of images using binary segmentation and binary alignment processor.

FIG. 5 shows the binary processor for binary lumen extraction.

FIG. 6 shows the Guidance Zone computation processor.

FIG. 7 shows the binary rigid alignment processor.

FIG. 8 shows the binary non-rigid alignment processor.

FIG. 9 shows the grayscale rigid alignment.

FIG. 10 shows the grayscale non-rigid rigid alignment.

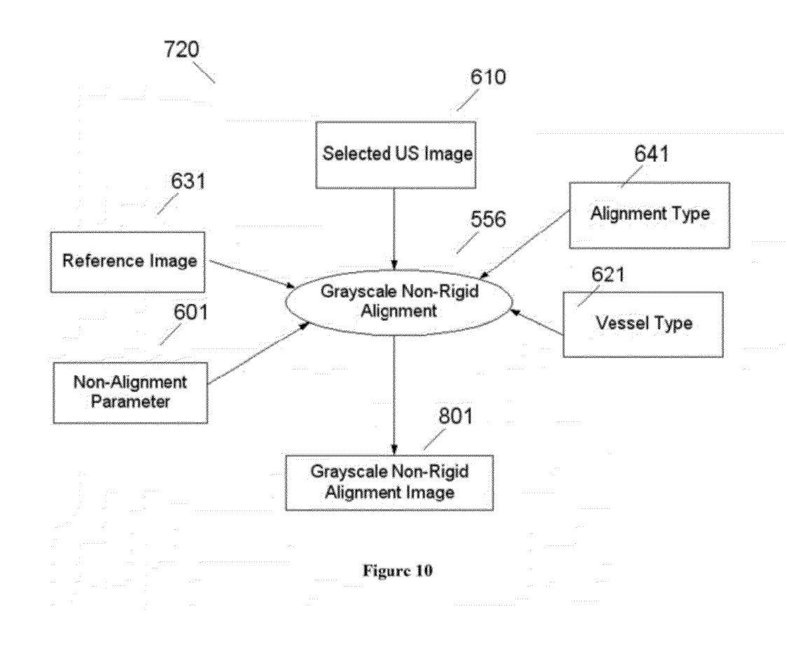

FIG. 11 shows the example showing the binary lumen region.

FIG. 12 shows another example of the binary lumen region.

FIG. 13 shows the example of the grayscale alignment.

FIG. 14 shows the mean brightness with highest far intensity.

FIG. 15 shows the mean brightness with highest far intensity.

FIG. 16 shows the IMT measurement process once identified the highest thr wall intensity. FIG. 16 (a) is the original grayscale image. FIG. 16 (b) shows the seed selection process using feature extraction retaining the seeds for the far wall. FIG. 16 (c) far wall media-adventitia (MA) borders. FIG. 16 (d) shows the adventitia borders for near and far wall.

FIG. 17 shows the entire process.

FIG. 18 shows the computer system.

### DETAILED DESCRIPTION

In the following description, for purposes of explanation, numerous specific details are set forth in order to provide a thorough understanding of the various embodiments. It will be evident, however, to one of ordinary skill in the art that the various embodiments may be practiced without these specific details.

As explained above, an algorithm localized the adventitial wall based on the intensity local maxima of every column in the image, i.e., the far wall brightness compared to the near wall. Several systems developed for IMT measurements that were based on this hypothesis produced good agreement with expert segmentation. The assumption was based on manual intensity measurements on several, representative US images.

Thus, the assumption that the far wall brightness is the highest intensity in the image can be used as a basis for automatically finding the far adventitia borders and then automatically using that as a marker for IMT measurement. This application is focused on (a) demonstrating that far wall has the highest brightness and then using this far wall brightness as a marker for automatically finding the for adventitia border and correspondingly its IMT measurement. The concept of validation this assumption or hypothesis used a novel approach to combine alignment of ultrasound images of blood vessels using a set of pre-computed alignment parameters. The pre-computed alignment parameters are computed using a novel alignment system which in turn uses regional information derived using segmentation criteria. Thus the validation system is a combination of segmentation and registration criteria, where the segmentation is driven by special speed functions as stoppers for deriving the binary regional information.

Another advantage of such a validation system where the highest brightness can be proven to be at far adventitia borders using the alignment parameters computed using regional information rather than point information provides estimation of robust alignment parameters.

Another advantage of such a validation system where the highest brightness can be proven to be at far adventitia borders using the alignment parameters computed using regional information rather than point information, where regional information is derived using level set controlled by a special speed function computed by subtracting every pixel in the image from the maximum value of the image and then multiplying the image by a function of the gradient of the original image.

Such a validation system is valid for arterial ultrasound images application for carotid, aorta, brachial and peripheral arteries. The validated hypothesis on highest brightness in the far wall adventitia region is then used for automated IMT estimation. Such a system is also used in a distributed architecture such as cloud-based system (AtheroCloud™), where the presentation layer is mobile system (AtheroMobile™) or a hand-held device and persistence layer is a server.

Another advantage of such a validation system where the highest brightness can be proven to be at far adventitia borders, can be used for automated far adventitia border detection and then can be used for LI and MA estimation process (AtheroEdge™), this region can then be used for tissue characterization for automated classification of the Atherosclerosis Disease to be symptomatic or asymptomatic (Atheromatic™).

Another advantage of such a validation system where the highest brightness can be proven to be at far adventitia borders, can be used for alignment of the arterial images over time and then use AtheroEdge™ system for measurement of IMT. Thus one can monitor the IMT over time (Atherometer™) to measure the effect of the therapy.

In this application, we validate that hypothesis of far wall maximum brightness by registering our entire database of ultrasound arterial images of the carotid artery and showing that the far wall has higher intensity in addition, we also look at the feasibility of automatic lumen segmentation and registration of B-mode or RF-mode US carotid artery images for clinical studies. Here, we are registering images to a ‘standard carotid artery’ but we can adapt the same method for follow up studies involving the same patient. After registration, we segment the images by an automated technique, in order to show the performance and exploit the potentialities of the registered images. The concept of joint registration and segmentation for hypothesis validation is the concept in this application.

This patent application discloses various embodiments of a computer-implemented system and method for fast, reliable and automated processing for validation of the highest brightness in the far wall of the blood vessel in ultrasound image and intima-media thickness (IMT) measurements. In particular, this patent application discloses various embodiments of a computer-implemented system and method for validation of highest brightness in the far wall of the carotid ultrasound image and its intima-media thickness (IMT) measurements. Although the embodiments disclosed herein are described in regard to particular blood vessels (e.g., carotid), the systems and methods disclosed and claimed are also applicable to validation and IMT measurement in any blood vessel in any living organs or tissue. For example, various embodiments can be used for validation and IMT measurement in carotid, femoral, brachial and aortic arteries. The details of various example embodiments are provided herein.

### Overview of Various Embodiments—

In the various example embodiments described herein, a variety of benefits and advantages are realized by the disclosed systems and methods. A representative sample of these advantages is listed below.

A validation processor that validates the far wail of the ultrasound blood vessel has the highest brightness in the image and hence can be used for automated far wall IMT measurement.

A validation processor that uses the automated alignment parameter processor using alignment parameters computed from segmentation criteria, and then using alignment parameters for grayscale alignment used for validation of highest brightness in the for wall of the carotid ultrasound image.

An automated binary alignment parameter processor that automatically segments the lumen region in the common carotid artery and generates the alignment parameters by aligning the binary lumen region with respect to binary lumen region as a reference image.

An automated binary alignment parameter processor that automatically segments the lumen region in the common carotid artery, where the lumen region is segmented utilizing the concept of multi-resolution framework.

An automated binary alignment parameter processor that automatically segments the lumen region in the common carotid artery, where the lumen region is segmented using multi-resolution framework in the guidance zone.

An automated binary alignment parameter processor that automatically segments the lumen region in the guidance zone, using the combination of level sets and mathematical morphology.

An automated binary alignment parameter processor that automatically segments the lumen region in the guidance zone, using the combination of level sets and mathematical morphology, where the level set is controlled by a special speed function that is computed by subtracting every pixel in the image from the maximum value of the image and then multiplying the image by a function of the gradient of the original image.

A validation processor that uses the aligned grayscale images to estimate the highest brightness in the far wall of the blood vessel in the ultrasound image.

A validation processor that uses the multi-resolution segmentation processor which uses a combination of fine to coarse resolution and recognition of far adventitia borders which is then used for guidance zone computation.

Another embodiment is the application of such a validation system for tour different kinds of arterial ultrasound images such as: carotid, aorta, brachial and peripheral arteries. The validated hypothesis on highest brightness in the far wall adventitia region is then used for automated IMT estimation. Such a system is also used in a distributed architecture such as cloud-based system (AtheroCloud™), where the presentation layer is mobile system (AtlleroMobile™) or a hand-held device and persistence layer is a server.

Another embodiment and advantage of such a validation system where the highest brightness can be proven to be at far adventitia borders, can be used for automated far adventitia border detection and then can be used for LI and MA estimation process (AtheroEdge™), this region can then be used for tissue characterization for automated classification of the Atherosclerosis Disease to be symptomatic or asymptomatic (Atheromatic™).

Another embodiment and advantage of such a validation system where the highest brightness can be proven to be at far adventitia borders, can be used for alignment of the arterial images over time and then use AtheroEdge™ system for measurement of IMT. Thus one can monitor the IMT over time (Atherometer™) to measure the effect of the therapy.

Another embodiment and advantage of such a validation system where the highest brightness can be proven to be at far adventitia borders, can be used for alignment of the arterial images over time and then used for a design of a vascular analysis system such as VesselOmeasure™ which is a hybrid or amalgamation of systems like AtheroEdge™, Atheromatic™, Atherometer™, AtheroRisk™.

### Detailed Description of Example Embodiments

This patent application discloses various embodiments of a computer-implemented system and method for fast, reliable and automated validation of the highest brightness of the far wall of the ultrasound blood vessel image and the corresponding intima-media thickness (IMT) measurements. In particular, this patent application discloses various embodiments of a computer-implemented system for validation of the highest brightness in the far wall and method for intima-media thickness (IMT) measurements. Although the embodiments disclosed herein are described in regard to particular blood vessels (e.g., carotid), the systems and methods disclosed and claimed are also applicable to brightness validation and IMT measurement in any blood vessel in any living organs or tissue. For example, various embodiments can be used for validation of highest brightness and IMT measurement in carotid, femoral, brachial and aortic arteries. The details of various example embodiments are provided herein.

IMT measurement is a very important risk marker of the Atherosclerosis disease. Typically, there are two ways to measure the arterial IMT's: (a) invasive methods and (b) non-invasive methods. In invasive methods, traditionally, intravascular ultrasound (IVUS) is used for measuring vessel wall thickness and plaque deposits where special catheters are inserted in the arteries to image them. Conventional ultrasound is used for measuring IMT non-invasively, such as from carotid, brachial, femoral and aortic arteries. The main advantages of non-invasive methods are: (i) low cost; (ii) convenience and comfort of the patient being examined; (iii) lack of need for any intravenous (IV) insertions or other body invasive methods (usually), and (iv) lack of any X-ray radiation; Ultrasound can be used repeatedly, over years, without compromising the patient's short or long term health status. Though conventional methods are generally suitable, conventional methods have certain problems related to accuracy, speed, and reliability. Further, the automated IMT methods suffer from the challenge that they hypothesize that the far wall has the brightest intensity.

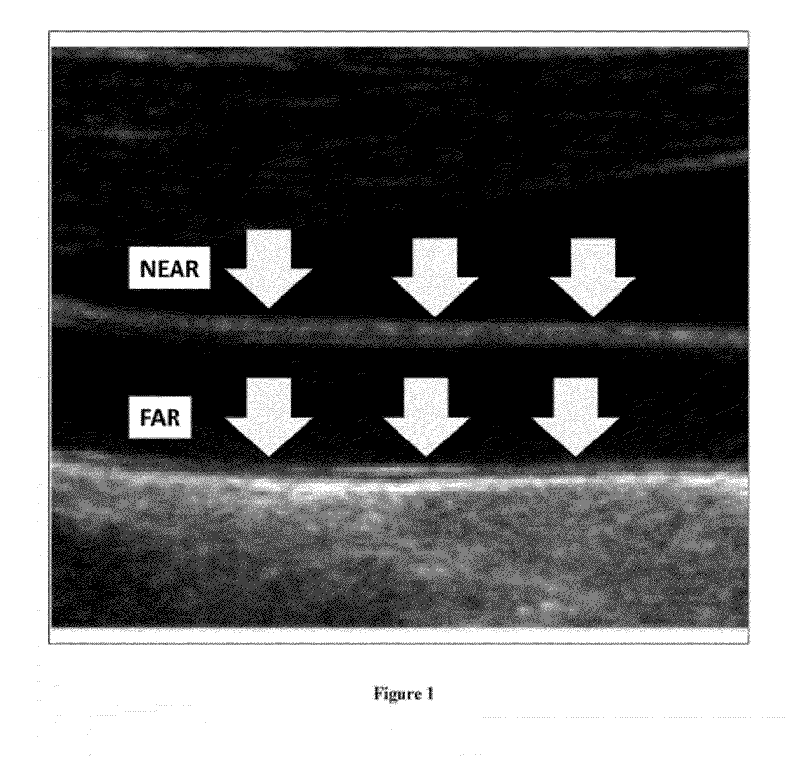

The IMTs are normally 1 mm in thickness, which nearly corresponds to 15 pixels on a typical screen or display. IMT estimation having a value close to 1 mm is a very challenging task in ultrasound images due to large numbers of variabilities, such as: poor contrast, orientation of the vessels, varying thickness, sudden fading of the contrast due to change in tissue density, presence of various plaque components in the intima wall such as fibrous muscles, lipids, calcium, hemorrhage, etc. Under normal resolutions, a 1 mm thick media thickness is difficult to estimate using stand-alone image processing techniques. Over and above, the image processing algorithms face an even tighter challenge due to the presence of speckle distribution. The speckle distribution is different in nature from these interfaces. This is because of the structural information change between intima, media and adventitia layers of the vessel wall. As a result, the sound reflection from different cellular structures is different. The variability in tissue structure—all that happens in 1 mm of the vessel wall—brings fuzziness in the intensity distribution of the vessel wall. Under histology, media and adventitia walls are clearly visible and one can observe even their thicknesses. This 1 mm zone is hard to discern in a normal resolution image of 256×256 pixels in a region of interest (ROI) or in a higher resolution image of 512×512 pixels in a region of interest (ROI). For automated IMT measurement, one needs a high resolution image to process and identify the intensity gradient change in ultrasound images from lumen to intima and media to adventitia layers. The ultrasound image resolution may not be strong enough like magnetic resonance imaging (MRI) or computerized axial tomography (CAT or CT) images, which can be meaningful for soft tissue structural information display. Further, automated IMT measurements systems hypothesize that far wall has the highest brightness in the image and hence the IMT measurement is being computed for the brightness wall. This application thus validates that the far wall of the blood vessel in the ultrasound image has the highest or brightest intensity. FIG. 1 shows the grayscale image of the carotid ultrasound showing the near and far walls of the carotid artery.

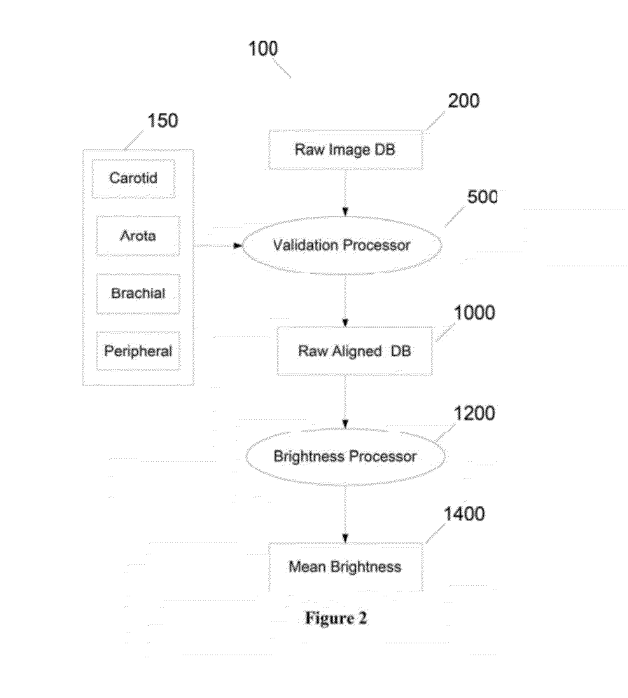

There are two ways to process and identify the intensity gradient change in ultrasound images from lumen to intima (LI) and media to adventitia (MA) layers: (a) have a vascular surgeon draw the LI/MA borders and compute the IMT image interactively, OR (b) have a computer determine the LI and MA borders along with IMT's. Case (a) is very subjective and introduces variability in the IMT estimation. IMT screenings are really part of the regular check-up for patients and millions of scans are done each day around the world. The manual handling of such a repetitive work flow of IMT screenings is tedious and error-prone. Case (b) is difficult to implement, because it is difficult to identify the LI and MA borders with heavy speckle distribution and the inability of ultrasound physics to generate a clear image where the semi-automated or automated image processing methods are used for IMT estimation. Besides that, the calcium deposit in the near walls causes the shadow. Most of the automated systems for IMT measurement hypnotizes that the far wall has the highest intensity and hence can be used as a marker for automated IMT measurement for the far all. This application validates this assumption. Once validated, can then used for automated IMT measurement, classification of the Atherosclerosis Disease into symptomatic type or asymptomatic type in the IMT wall region (Atheromatic™). Another advantage of such a validation system can be for the usage of IMT estimation (AtheroEdge™). Another advantage of such a hypothesis validation system is to monitor IMT region around the highest brightness region (Atherometer™). Another advantage of such a brightness far wall validation system is to use this in the IMT measurement in a distributed architecture nature where the presentation layer is hand-held device and, persistence layer is the cloud or server for mobile applications (AtheroMobile™). FIG. 2 shows the validation processor. The object 200 is the raw input image database which is input to the validation processor 500. The output of the validation processor is the raw aligned image database given the grayscale reference image. The reference image must be of same anatomic region as the image database. Though this application uses carotid ultrasound, but this is application to all the arterial images such as carotid, aorta, brachial and peripheral. This means if the ultrasound image database is for common carotid blood vessels, then the reference image must correspond to the carotid common carotid artery. Similar analogy is applied for brachial, aorta and peripheral. If the ultrasound image database is for aortic images, then the reference image must also correspond to the aortic artery. This is shown in the block 150. Brightness processor 1200 is then used on the raw aligned database to display the 3D plot for the mean brightness for validation.

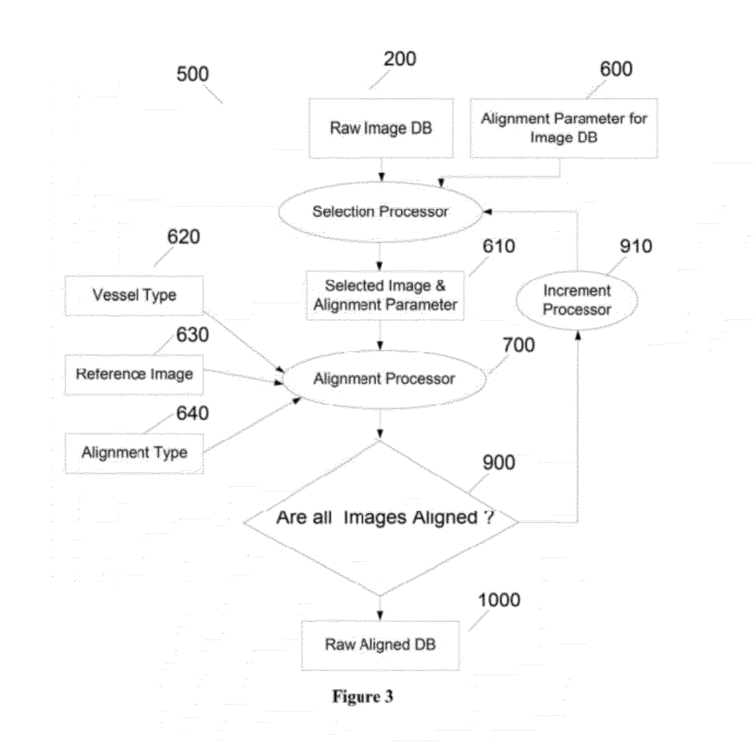

FIG. 3 shows the OPD (object process diagram) for the validation processor 500. Given the raw image database 200 and the alignment parameters 600 for the database, the system processes each grayscale raw image and aligns with the grayscale reference image 630. The selection processor selects the grayscale raw image and the corresponding alignment parameter and yields the selected raw image and its corresponding alignment parameter 610. The raw grayscale image is then aligned with the reference image 630) using the alignment parameter 600. The alignment processor 700 uses these three sets of information to give the aligned image 1000 and put in the aligned database 1000. If the validation processor has any unprocessed images, the process continues by incrementing to next image using the block 910. The selection processor then further selects the grayscale raw image, corresponding alignment parameter corresponding to this image and the grayscale raw reference image 630. Note that this validation processor is used for carotid ultrasound application but is applicable to aorta, brachial, and peripheral arteries. This is accomplished using the flag 620 called vessel type. This can take four different options: if Flag is set to 1, the carotid artery option is choose, if the flag is set to 2, aorta option is chosen, if the flag is set to 3, brachial artery is choose and if the flag is set to 4, peripheral option is chosen. Those skilled in the art can use this validation processor for coronary artery application where the atherosclerotic plaque is deposited. The alignment processor 700 also uses an option 640 to select if the process is a rigid alignment or a non-rigid alignment. The key innovation in this application for validation for the brightest wall in the carotid ultrasound artery is usage of alignment parameter which is obtained using the binary regional segmentation processor. Thus the validation processor is driven by the combination of a “segmentation and alignment” processors. This provides an advantage to the system to change the artery if the segmentation process needs to be changed, which in turn changes the alignment parameters 600. Note that the validation processor 500 can be an online processor given the alignment parameters 600. The block 1000 shows the final output of the validation processor which is then used for mean brightness computation for validation of the highest brightness in the carotid artery far wall ultrasound image. The highest brightness far wall is then used for feature extraction which is then used for LI and MA border estimation, which then is used for IMT estimation (called AtheroEdge™ system). Another advantage for finding highest brightness and validating highest brightness for far wall is for computing features which is then used for estimating the nature of the Atherosclerosis Disease such as symptomatic and asymptomatic type (called. Atheromatic™ system). Another advantage of this system is usage of the alignment parameters for alignment of arterial ultrasound images for monitoring the IMT and its changes (called Atherometer™ system). This can track the changes in IMT over time to study the drug effect. Further the system allows integration of the AtheroEdge™, Atheromatic™, Atherometer™ into a vascular analysis system (VesselOmeasure™).

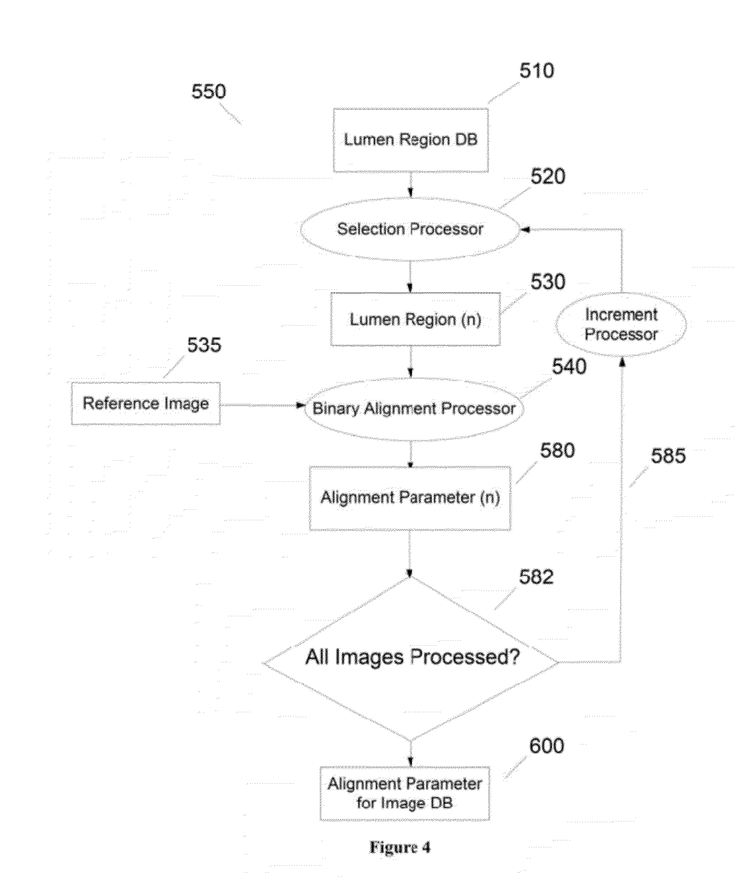

FIG. 4 shows the processor 550 which shows the alignment parameters 600 for the input database. Given the binary lumen region 510 of the common carotid artery in the carotid ultrasound image, the processor 550 allows to obtain the alignment parameter. The reference binary image 535 is used as a reference for computing the alignment parameter for the given image (n). The block 510 is the image database of binary lumen regions which is then used for computing the alignment parameters 600 (FIG. 3). The process of computing the alignment parameters consists of binary lumen region image selection 520 to give the binary lumen region image 530. This binary lumen region CCA image is then aligned with given binary lumen region reference image 535 using the binary alignment processor 540, yielding the output alignment parameter in) 580. The process of binary alignment is repeated as shown in the loop 585. The final output is obtained and stored in the alignment parameter database 600. Note alignment parameter processor 550 is applicable for any artery such as carotid, aorta, brachial and peripheral. Those skilled in the art can use the same paradigm and concept for coronary artery ultrasound images. The key innovation in this process is the role of creation of alignment parameter database 600 which is then used for validation of far wall highest brightness in the carotid ultrasound image. This validation processor 600 then uses the off-line alignment parameter database which uses the combination of “segmentation and alignment” for creating the alignment database. The main advantage of the system is the usage of alignment parameters which are based on regional information rather than point-based information for alignment. This provides another layer of robustness to the validation system. These binary regional alignment parameters are then used for alignment of the images with respect to reference image and such information can then be also used for monitoring the IMT changes over time such as seeing the effect of drug therapy (Atherometer™).

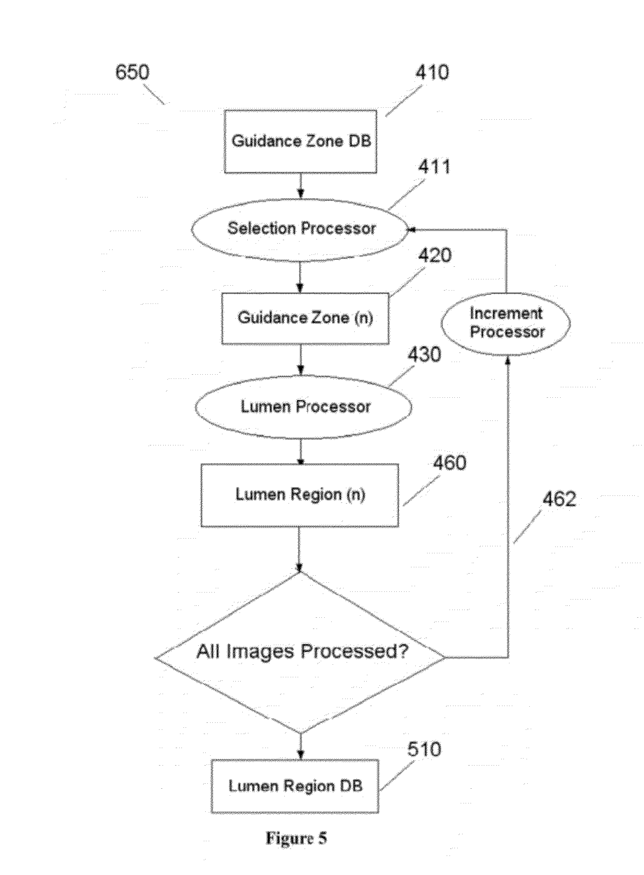

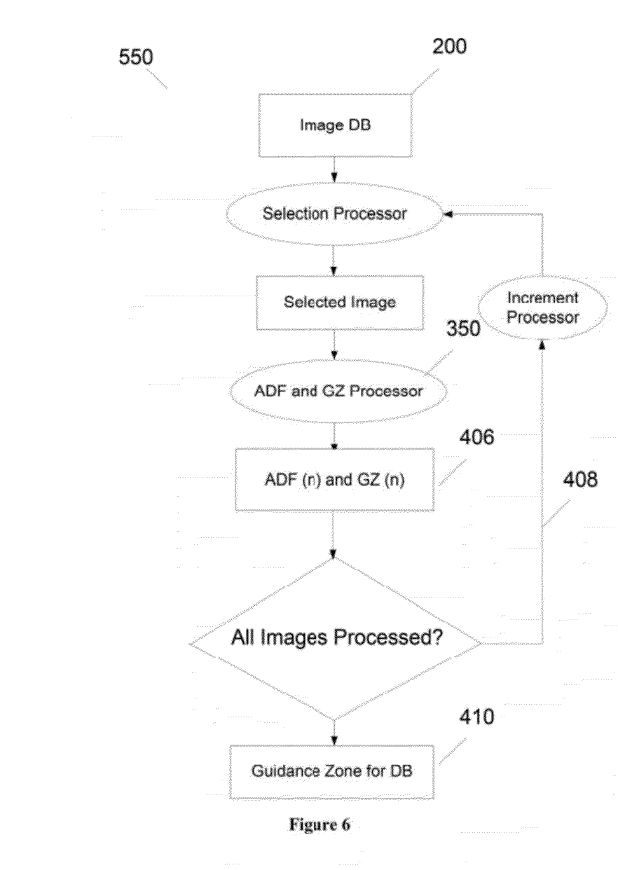

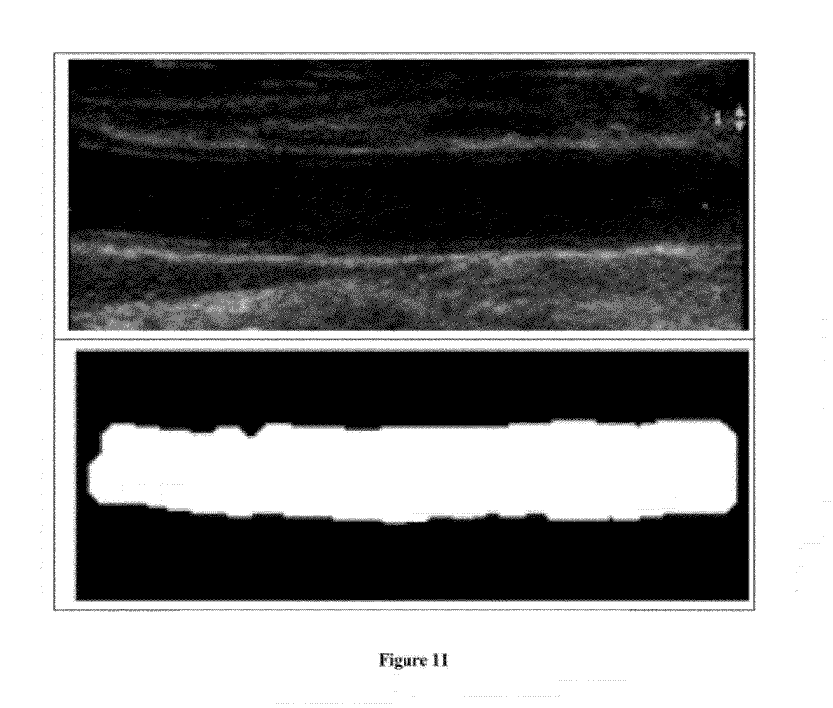

The binary lumen region 510 is obtained by processing the grayscale guidance zone region 410 as shown in the FIG. 5 (block 650). Guidance zone is a rectangular region or envelope which takes into consideration the near and far wall and the lumen region between them. This guidance zone allows to create a binary lumen region 510 which is then used for binary alignment processor 540 (FIG. 4). The process for binary lumen region database creation is shown in FIG. 5. Given the Guidance Zone DB 410, image selection processor 411 selects the Guidance Zone (n) 420, which is then used for lumen region extraction 460 using the lumen processor 430. The process is repeated as shown in block 462 till all the Guidance Zone images of block 410 is completed. The process of Guidance Zone creation is shown in FIG. 6. FIG. 11 shows the binary region extracted by the lumen processor and used for binary alignment.

The process of Guidance Zone creation is shown in FIG. 6. This Guidance Zone (GZ) is computed from the original raw image and creates automatically the Guidance Zone. This patent application uses a multi-resolution system for computing for adventitia borders of the arterial wall and building GZ around it. The GZ is computed for each image (n). This is repeated using the process by incrementing using the process 408.

### Steps for Guidance Zone (GZ) Estimation:

Multi-resolution image processing consists of down sampling the image from fine to coarse resolution. One of four systems can be used for fine to coarse sampling. The role of the multi-resolution process is to convert the image from fine resolution to coarse resolution. Those of ordinary skill in the art of down sampling can use any off-the-shelf down sampling methods. One of the very good down samplers is Lanczos interpolation. This is based on the sine function which can be given mathematically as:

$$sin c ( x ) = \frac{sin ( π x )}{π x} .$$

Because the sine function never goes to zero, a practical tilter can be implemented by taking the sine function and multiplying it by a “window”, such as Hamming and Hann, giving an overall filter with finite size. We can define the Lanczos window as a sine function scaled to be wider, and truncated to zero outside of the main lobe. Therefore, the Lanczos filter is a sine function, multiplied by a Lanczos window. A three lobed Lanczos filter can be defined as:

$$Lanczos 3 ( x ) = \{ \begin{matrix}\frac{sin ( π x ) sin ( π \frac{x}{3} )}{π x \cdot π \frac{x}{3}} , & if \vert x \vert \leq 3 \\ 0 , & if \vert x \vert > 3\end{matrix}$$

Although Lanczos interpolation is slower than other approaches, it can obtain the best interpolation results; because, the Lanczos method attempts to reconstruct the image by using a series of overlapping sine waves to produce what's called a “best fit” curve. Those of ordinary skill in the art of image down sampling, can also use Wavelet transform filters as they are very useful for multi-resolution analysis. In a particular embodiment, the orthogonal wavelet transform of a signal f can be formulated by:

$$f ( t ) = \text{?} ( k ) {ϕ}_{j , k} ( t ) + \text{?} {d}_{j} ( k ) {ϕ}_{j , k} ( t )$$

$$\text{?} \text{indicates text missing or illegible when filed}$$

where the cⱼ(k) is the expansion coefficients and the dⱼ(k) is the wavelet coefficients. The basis function φ_{j,k}(t) can be presented as:

φ_{j,k}(t)=2^{-j/2}φ(2^{-j}t−k),

where k, j are translation and dilation of a wavelet function φ(t). Therefore, wavelet transforms can provide a smooth approximation of f(t) at scale J and a wavelet decomposition at per scales. For 2-D images, orthogonal wavelet transforms will decompose the original image into four different sub-bands (LL, LH, HL and HH).

Bi-cubic interpolation can also be used, as it will estimate the value at a given point in the destination image by an average of 16 pixels surrounding the closest corresponding pixel in the source image. Given a point (x,y) in the destination image and the point (l,k) (the definitions of l and k are the same as the bilinear method) in the source image, the formulae of bi-cubic interpolation is:

$$f ( x , y ) = \text{?} g ( m , n ) \cdot r ( m - l - dx ) \cdot ( dy - n + k ) , \\ \text{?} \text{indicates text missing or illegible when filed}$$

where the calculation of dx and dy are the same as the bilinear method. The cubic weighting function r(x) is defined as:

$$r ( x ) = \frac{1}{6} [ {p ( x + 2 )}^{3} - 4 {p ( x + 1 )}^{3} + 6 {p ( x )}^{3} - 4 {p ( x - 1 )}^{3} ] ,$$

where p(x) is:

$$p ( x ) = \{ \begin{matrix}x & x > 0 \\ 0 & x \leq 0\end{matrix}$$

The bi-cubic approach can achieve a better performance than the bilinear method; because, more neighboring points are included to calculate the interpolation value.

A bilinear interpolator can also be used as it is very simple to implement. Mathematically, a bilinear interpolator is given as: if g represents a source image and f represents a destination image, given a point (x,y) in f, the bilinear method can be presented as:

$$f ( x , y ) = ( 1 - dx ) \cdot ( 1 - dy ) \cdot g ( l , k ) + dx \cdot ( 1 - dy ) \cdot g ( l + 1 , k ) + ( 1 - dx ) \cdot dy \cdot g ( l , k + 1 ) + dx \cdot dy \cdot g ( l + 1 , k + 1 ) ,$$

where l=└x┘ and k=└y┘, and the dx, dy are defined as dx=x−l and dy=y−k respectively. Bilinear interpolation is simple. However, it can cause a small decrease in resolution and blurring because of the averaging nature of the computation.

The next step consists of the de-speckle filtering. Speckle noise was attenuated by using a first-order statistics filter, which gave the best performance in the specific case of carotid imaging. This filter is defined by the following equation:

J_{x,y}=Ī+k_{x,y}(I_{x,y}−Ī) (1)

where, I_{x,y} is the intensity of the noisy pixel, Ī is the mean intensity of a N×M pixel neighborhood and k_{x,y} is a local statistic measure. The noise-free pixel is indicated by J_{x,y}, k_{x,y} is mathematically defined as:

$${k}_{x , y} = \frac{{σ}_{l}^{2}}{{\overline{I}}^{2} {σ}_{l}^{2} + {σ}_{n}^{2}} ,$$

where σ_{J}² represents the variance of the pixels in the neighborhood, and σ_{K}² the variance of the noise in the cropped image. An optimal neighborhood size in an example embodiment can be 7×7 pixels. Note that the despeckle filter is useful in removing the spurious peaks, if any, during the adventitia identification in subsequent steps. Those of ordinary skill in the art can use any local statistical noise removal filter or filters based on morphological processing or filters presented in Suri et al., MODELING SEGMENTATION VIA GEOMETRIC DEFORMABLE REGULARIZERS, PDE AND LEVEL SETS IN STILL AND MOTION IMAGERY: A REVISVIT. International Journal of Image and Graphics, Vol. 1, No. 4 (2001) 681-734.

After down sampling and despeckling, one can do the far adventitia determination using the convolution of the first order derivative. The scale parameter of the Gaussian derivative kernel was taken equal to 8 pixels, i.e. to the expected dimension of the IMT value. In fact, an average IMT value of say 1 mm corresponds to about 16 pixels in the original image scale and, consequently, to 8 pixels in the coarse or down sampled image. The convolution processor outcome will lead to the clear information for the near and far vessel walls. This information will have two parallel bands corresponding to the far and near vessel walls. These bands will follow the curvature of the vessel walls. If the vessel wall is oriented downwards or upwards or has a bending nature, the bands will follow on both sides of the lumen. These bands have information which corresponds to the maximum intensity saturated to the maximum values of 2 powers 8, the highest value. For an 8 bit image, this value will be 255.

The convolution process then allows the heuristics to estimate the Far Adventitia borders of the far wall or near wall. To automatically trace the profile of the far wall, the processor uses the heuristic search applied to the intensity profile of each column. In a particular embodiment, we use an image convention wherein (0,0) is the top left hand corner of the image. Starting from the bottom of the image (i.e., from the pixel with the higher row index), the processor searchers for the first white region constituting at least 6 pixels of width. The deepest point of this region (i.e., the pixel with the higher row index) marked the position of the far adventitia (AD_{F}) layer on that column. The sequence of points resulting from the heuristic search for all the image columns constitutes the overall automated far wall adventitia tracing AD_{F}.

The last stage of the Artery Recognition Processor is the up-sampling processor which allows the adventitia tracing AD_{F} to be up-sampled back to the original scale of cropped image. The AD_{F} profile was then up-sampled to the original scale and superimposed over the original cropped image for both visualization and determination of the Guidance Zone for the binary segmentation of the lumen region which is then used for binary alignment. At this stage, the CA far wall is automatically located in the image frame and automated segmentation is made possible. Then the Guidance Zone can be reconstructed from this ADF border. It is in this region, one can find the lumen which is the used for binary alignment.

### Summary of Binary Lumen Segmentation Steps:

Subsequently, our procedure automatically recognized the carotid artery in the image. We adopted a multi-resolution approach consisting of the following steps:

(1) Downsampling: The image is downsampled by a factor of 2 and speckle noise was attenuated. This scaled the size of the carotid wall (nominally about 1 mm=about 16 pixels) to the optimal size (8 pixels) for the automated recognition.
(2) Convolution with Higher Order Derivative: The image is filtered by using a first-order derivative Gaussian filter. This filter is the equivalent of a high-pass filter, which enhances the representation of the objects having the same size of the kernel, i.e., 8 pixels.
(3) Heuristic Search for AD_{F}: Starting from the bottom of the image, the far carotid wall was recognized as it was a bright stripe of about 8 pixels size. As recalled in step 1, since the nominal value of the IMT is about 1 mm, it is equivalent to 8 pixels in the down sampled domain. Thus, the first-order Gaussian derivative kernel is size matched to the IMT and it outputs a white stripe of the same size of the far wall thickness. The heuristic search considered the image column-wise. The intensity profile of each column was scanned from bottom to top (i.e. from the deepest pixel moving upwards). The deepest region which had a width of at least 8 pixels was considered as the far wall.
(4) Guidance Zone Creation: The output of this carotid recognition stage was the tracing of the far adventitia layer (AD_{F}). We then selected a Guidance Zone (GZ) in which we performed binary segmentation. The basic idea was to draw a GZ that comprised the far wall (i.e., the intima, media, and adventitia layers) and the near wall. The average diameter of the carotid lumen is 6 mm, which roughly corresponded to 96 pixels at a pixel density of 16 pixels/mm. Therefore, we traced a GZ that had the same horizontal support of the AD_{F} profile, and a vertical height of about 200 pixels. With this vertical size, which is double the normal size of the carotid, we ensured the presence, in the GZ, of both artery walls.
(5) Lumen Segmentation: The lumen segmentation consists of a preprocessing step followed by a level set based segmentation method. The first preprocessing step is the inversion of the image i.e. we subtract every pixel in the image from the maximum value of the image. We then multiply the image by a function of the gradient of the original image given by Eqn. (1) below.

$$f ( ∇ u ) = \frac{1}{1 + \vert ∇ u \vert} \qquad ( 1 )$$

Here u indicates the image. The function is such that it takes low values (<−1) at the edges in the image and tends to a maximum value of 1 in regions that are ‘flat’. The lumen is then segmented using the active contour method. Those skilled in the art can use any active contour method such as active contour without edges algorithm or the Chan-Vese algorithm. The algorithm was chosen based on the piecewise constant nature of the cropped carotid artery images obtained from the previous step. The lumen after pre-processing is white and its grayscale intensity is high (>100) with noise. The walls of the artery that initially appear bright becomes dark (i.e. <50 gray scale value) after preprocessing. The Chan-Vese method is very effective for segmenting images made up to two piecewise constant regions which in our case correspond to the lumen and the carotid wall. We formulate the segmentation problem as an energy minimization problem to find the optimum curve segmenting the regions, the energy term to be minimized can be written as

$$E = \text{?} {\vert {u}_{0} - {c}_{1} \vert}^{2} d x d y + \text{?} {\vert {u}_{0} - {c}_{2} \vert}^{2} d x d y \\ \text{?} \text{indicates text missing or illegible when filed} \qquad ( 2 )$$

where cᵢₙ and cₒᵤₜ refer to the regions enclosed by the optimum curve C that separates the two regions in the image u₀. The terms c₁ and c₂ are the average values of the two regions. We can also add regularization terms that are proportional to the length of the curve (L_{c}) and the area of the curve (A_{c}).

$${λ}_{1} \text{?} {\vert {u}_{0} - {c}_{1} \vert}^{2} d x d y + {λ}_{2} \text{?} {\vert {u}_{0} - {c}_{3} \vert}^{2} d x d y + \text{?} + μ {L}_{c} \\ \text{?} \text{indicates text missing or illegible when filed} \qquad ( 3 )$$

Here μ, ν, λ₁ and λ₂ are fixed parameters. The level set formulation is obtained by replacing the curve C with a level set function φ such that C is the level set with value 0. The function φ takes values less than zero inside the contour and positive values outside the contour. The energy is rewritten as

$${λ}_{1} {\int }_{Ω} {\vert {u}_{0} - {c}_{1} \vert}^{2} \text{?} ( φ ( x , y ) ) d x d y + {λ}_{2} {\int }_{Ω} {\vert {u}_{0} - {c}_{2} \vert}^{2} ( 1 - \text{?} ( φ ( x , y ) ) d x d y ++ μ {\int }_{Ω} \text{?} \vert ∇ φ ( x , y ) \vert d x d y \\ \text{?} \text{indicates text missing or illegible when filed} \qquad ( 4 )$$

where H_{c} is the regularized version of the Heaviside function given by Eqn. (5).

$$\text{?} ( z ) = 1 , z > ɛ \\ \text{?} ( z ) = 0 , z < - ɛ \\ \text{?} ( z ) = \frac{1}{2} [ 1 + \frac{z}{ɛ} + \frac{1}{π} sin ( \frac{π z}{ɛ} ) ] , \vert z \vert \leq ɛ \\ \text{?} \text{indicates text missing or illegible when filed} \qquad ( 5 )$$

We used a value of 10e-5 for ε. The Heaviside function is defined as 1 if its argument is non-negative and 0 otherwise. The derivative of the Heaviside function is the delta function (δ_{ε}). Ω is the domain of the level set function. The associated Euler-Lagrange equation for φ is given by Eqn. (6) below

$$\frac{\partial φ}{\partial t} = \text{?} ( φ ) [ μ div ( \frac{∇ φ}{\vert ∇ φ \vert} ) - {λ}_{1} \cdot {( {u}_{0} - {c}_{1} )}^{2} + {λ}_{2} \cdot {( {u}_{0} - {c}_{2} )}^{2} ] \\ \text{?} \text{indicates text missing or illegible when filed} \qquad ( 6 )$$

The boundary conditions are

$$\frac{\text{?} ( φ )}{\vert ∇ φ \vert} \frac{\partial φ}{\partial n} = 0 , \partial Ω \\ \text{?} ( 0 , x , y ) = \text{?} ( x , y ) \\ \text{?} \text{indicates text missing or illegible when filed} \qquad ( 7 )$$

The equation is discretized and solved numerically. The segmentation produces a binary image where the lumen is white (intensity of 1) and the wall intensity is 0. We would also like to point out that the algorithm is not influenced by the actual gray scale values in the carotid images giving us the flexibility to analyze images acquired with different settings. The algorithm is also robust to noise as it does not directly depend on the edges in the images.

### Summary of Automated Segmentation Algorithm—

Summary of Automated Segmentation Algorithm.
A) Speed function design using Image Inversion i.e. subtract image from maximum value in the image
B) Edge Enhancement for Speed Control Function: Multiply with function given in Eqn.

$$i . e . , f ( ∇ u ) = \frac{1}{1 + \vert ∇ u \vert} , \qquad ( 1 )$$

C) Multiresolution PDF: Since numerical Partial Differential Equations require long computing times, we down sampled the image by four times to achieve convergence in a reasonable amount of time.
D) Level Set Initialization in Guidance Zone: Automated Initialize level set contour as a rectangle.
E) Level Set Iterations: Run the Chan-Vese algorithm for a fixed set of iterations, empirically computed. The number of iterations is empirically computed for a particular set of databases.

F) Upsampling: The segmentation result which is a binary image where the lumen is 1 is up sampled to the original size of the image.
G) Connected Component Analysis: The presence of the jugular vein in some images causes the algorithm to segment both the carotid and the vein. In this case a connected component analysis is done to determine which connected group is closer to the AD_{F}. The connected group closest to the AD_{F} is taken as the carotid artery.
H) Morphological Cleaning: Morphological Hole filling is done to remove holes in the binary segmentation result, caused by the backscatter noise in the lumen. This constitute the binary lumen region image which is the used for binary alignment, which is then used for validation processor.

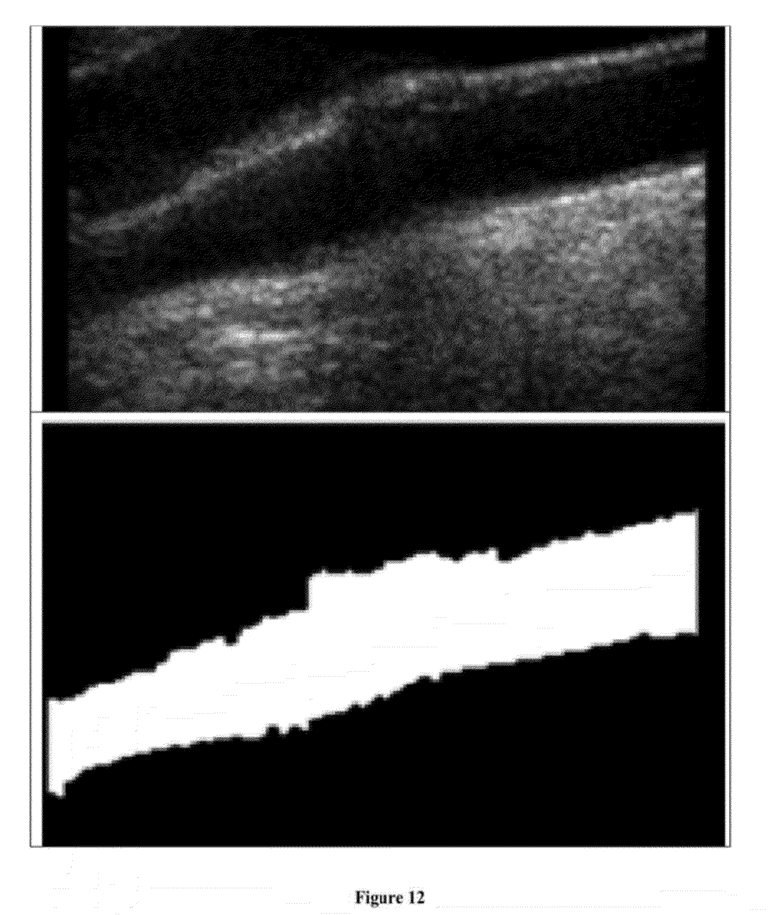

The main advantages of the binary lumen region extraction for rigid or non-rigid binary alignment are: (a) strong stopping force using multiplicative gradient in inverse image framework; (b) multi-resolution segmentation for high speed; (c) automated cleaning using a combination of connected components and morphology. The results of the accurate binary processor can be seen in the FIG. 11 and FIG. 12 for horizontal and inclined artery. Thus the system is robust for any slopped artery. Those of ordinary skill in the art can use a non-geometric deformable model for regional information extraction for binary alignment estimation.

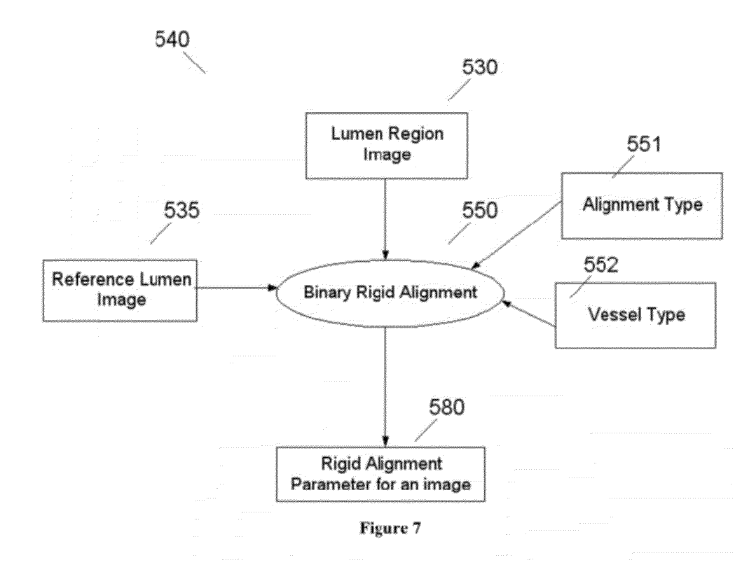

FIG. 7 shows the binary rigid alignment processor 540 (FIG. 4). The processor 540 is an example where the lumen region 530 is aligned using lumen reference binary image 535. The processor 550 is a binary rigid alignment process that aligns the binary lumen region 530 and binary lumen region 535 corresponding to the reference image.

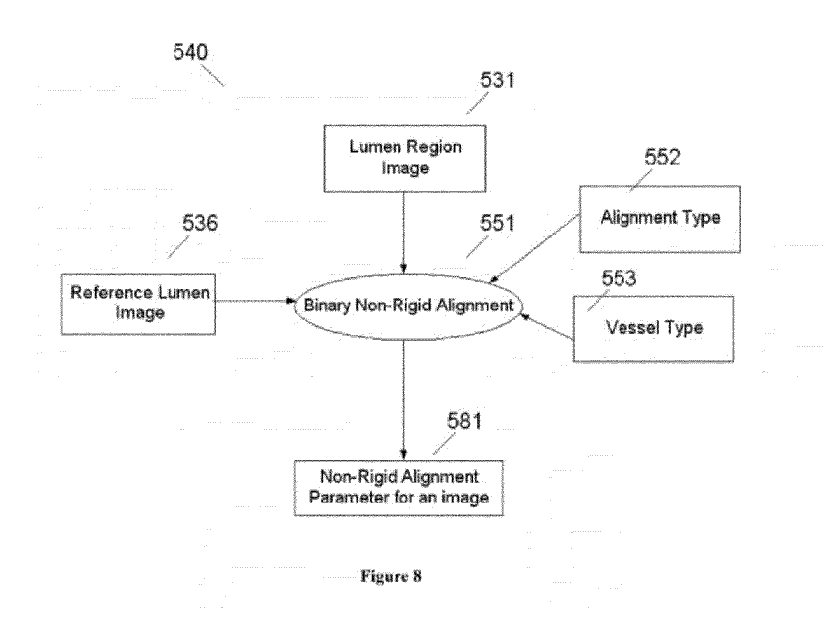

FIG. 8 shows the binary non-rigid alignment processor 540 (FIG. 4). The processor 540 is an example where the lumen region 530 is aligned using lumen reference binary image 536. The processor 551 is a binary non-rigid alignment processor that aligns the binary lumen region 531 and binary lumen region 536 corresponding to the reference image. Thus the system provides the advantage of using the rigid or a non-rigid binary alignment parameter estimation process using the regional information.

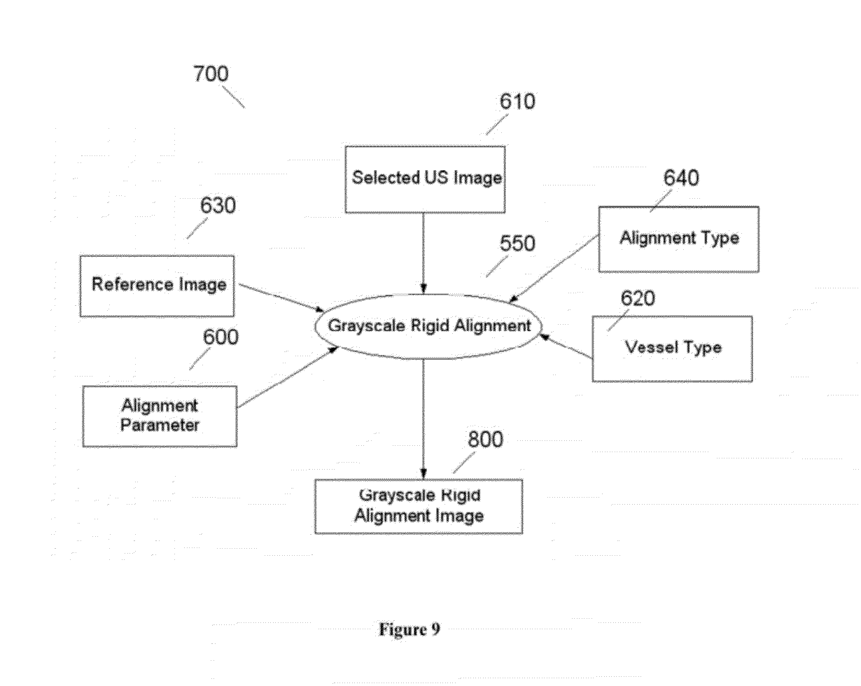

FIG. 9 shows the grayscale alignment processor 700 (from FIG. 3). The OPD for the processor inputs the selected grayscale raw carotid ultrasound image 610 and the reference image 610, along with the binary alignment parameter 600 and generates the grayscale rigid alignment image 800 using the grayscale rigid alignment processor 550. The system is using the alignment flag 640 and the vessel type flag 620. The alignment flag accepts the input rigid or non-rigid alignment flag. The vessel type flag allows to run either the carotid, aorta, brachial or peripheral blood vessel images.

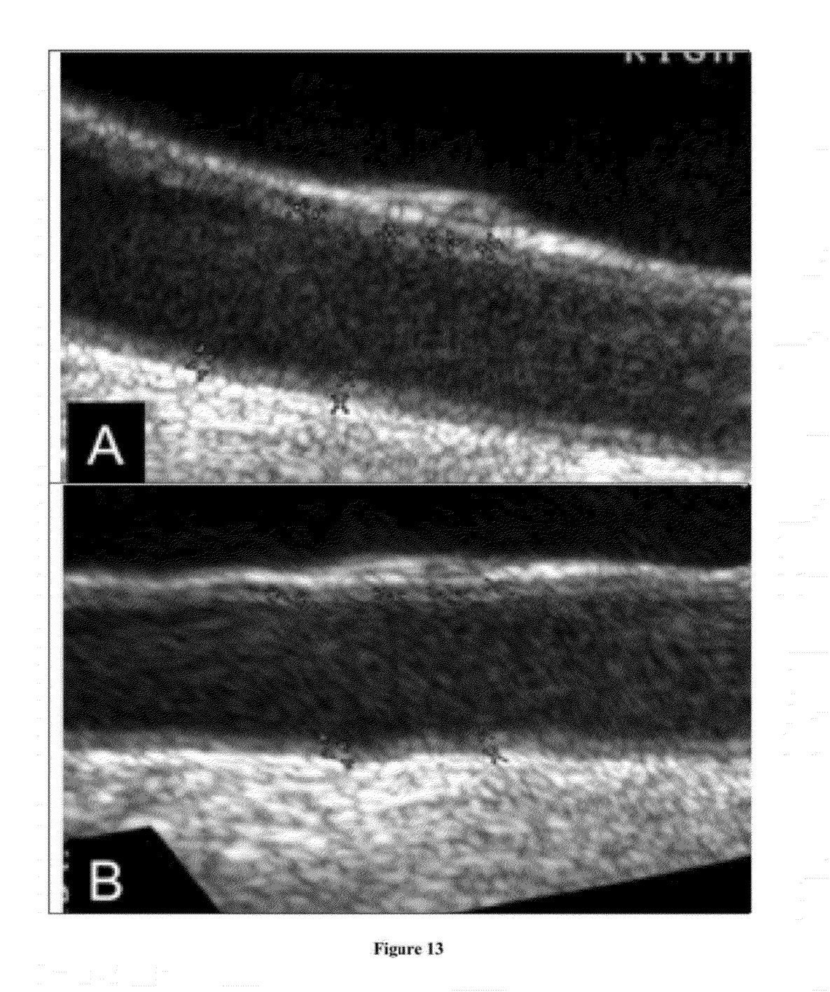

FIG. 13 shows an example input raw grayscale image and the corresponding aligned grayscale image. FIG. 13 (A) shows the unaligned image and FIG. 13 (B) shows the aligned image with respect to the reference image. Since the reference grayscale arterial image has common carotid artery nearly a straight vessel, thus the aligned image is transformed into a straight image via the transformation parameters, obtained from the binary alignment processor.

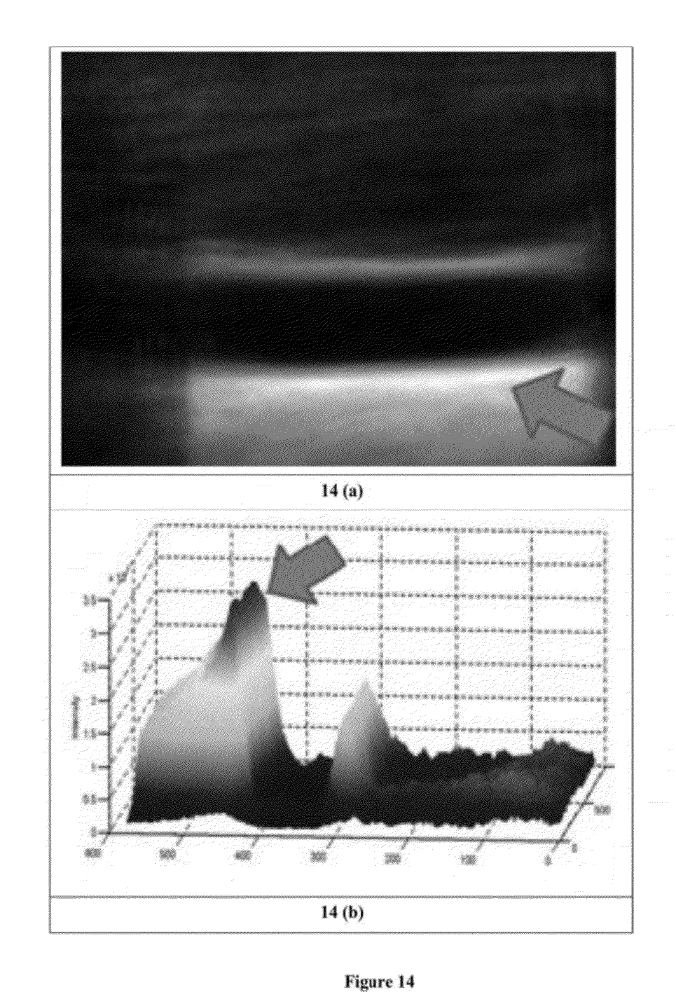

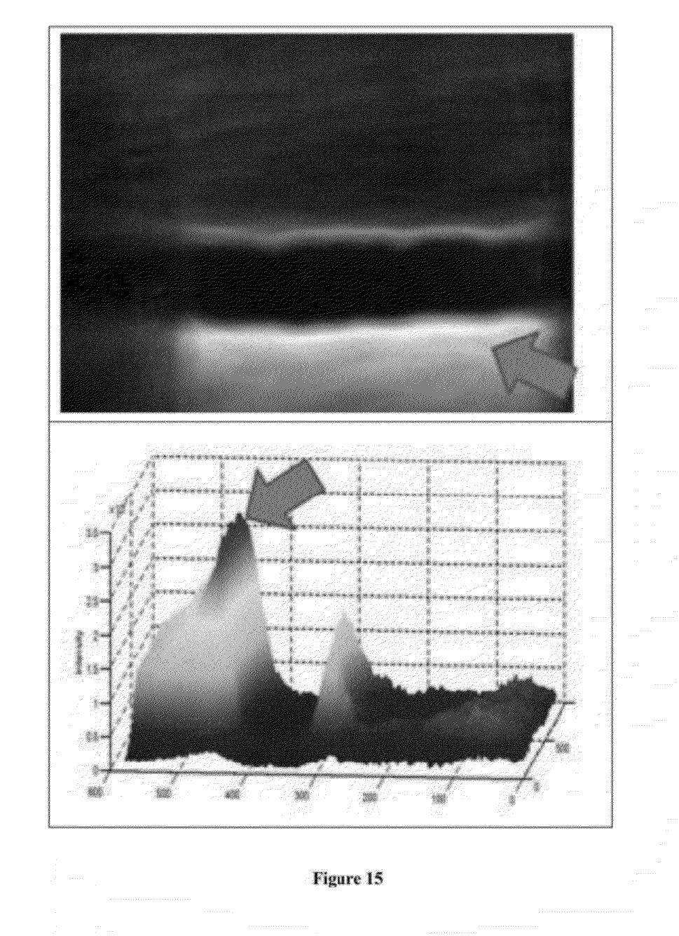

FIG. 14 shows the output of the validation processor. FIG. 14 (a) shows the mean brightness distribution after the alignment processor for all the images of the database. The mean intensity using a 3D plotter is shown in colored in FIG. 14 (b). The far wall shows the peak distribution. The corresponding information can be seen in FIG. 14 (a) as a bright thick line of white color showing an arrow demonstrating the system's ability to prove that the far wall has the highest brightness in the carotid ultrasound image. FIG. 15 shows a similar example using another database.

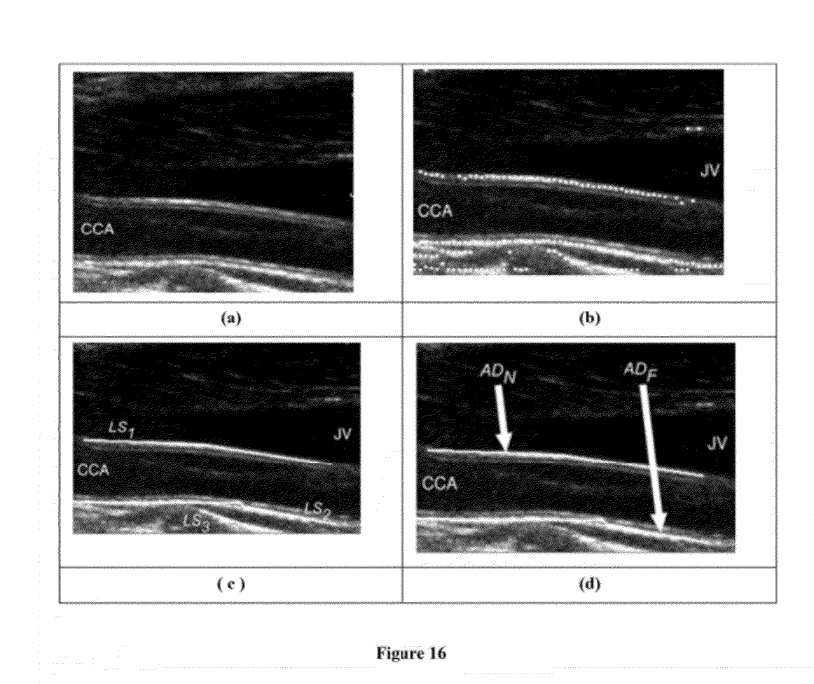

FIG. 16 shows the far adventitia computation for the far wall and the near wall. The seed points are selected in the far wall based on highest intensity of the far wall proven by the above validation processor. The validation processor that consists of a combination of a segmentation system and an alignment system. Such a system can then find the far and near adventitia borders. This can be further used for computation of IMT of the far wall. Thus the AtheroEdge™ like system can be used far IMT measurement once the hypothesis validation processor has estimated the highest brightness far all. FIG. 16 (a) is the original image, FIG. 16 (b) are the seed points based on intensity for the far and the near walls, FIG. 16 (c) is shows the connected lines from the seed points and FIG. 16 (d) is the final far adventitia lines for near and the far walls. This can then be used for Lumen Diameter measurement and IMT wall measurements as well. The feature-based method is used here to estimate the IMT measurement, given the far wall estimated by the validation processor.

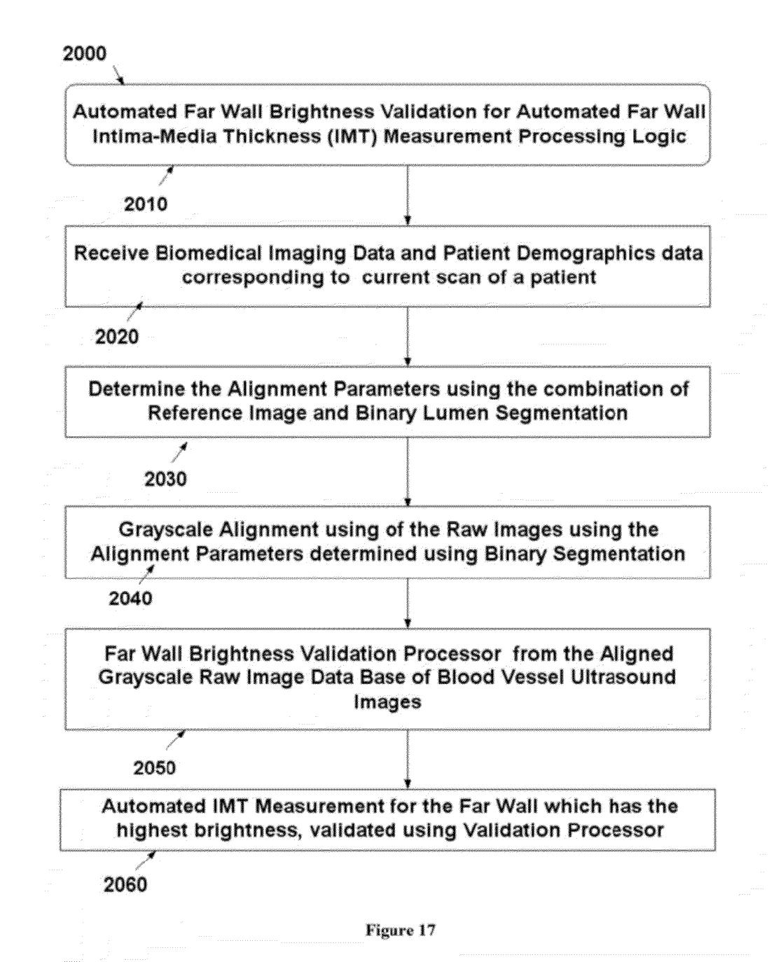

FIG. 17 is a processing flow diagram illustrating an example embodiment of a computer-implemented system and method for fast, reliable and automated validation system and processing of intima-media thickness (IMT) measurements as described herein. The method 2000 of an example embodiment includes validation processor and IMT computation (block 2010); select the biomedical imaging data (processing block 2020); computation of alignment parameters using a combination of segmentation and alignment processors (processing block 2030); alignment of the grayscale raw ultrasound images given the reference image (processing block 2040); mean brightness computation (processing block 2050); automated IMT computation (AtheroEdge™) given the far wall brightness (processing block 2060). Once the IMT region is estimated, the system can characterize the tissue in this region and support in decision making process for symptomatic and asymptomatic disease classification (Atheromatic™). Further, the system can be used for stroke risk prediction (AtheroRisk™) by tissue characterization in this IMT wall region. The block 2040 is used further for alignment of raw grayscale common carotid images for monitoring of the IMT over time or measuring IMT over time or metering IMT over time (called Atherometer™).

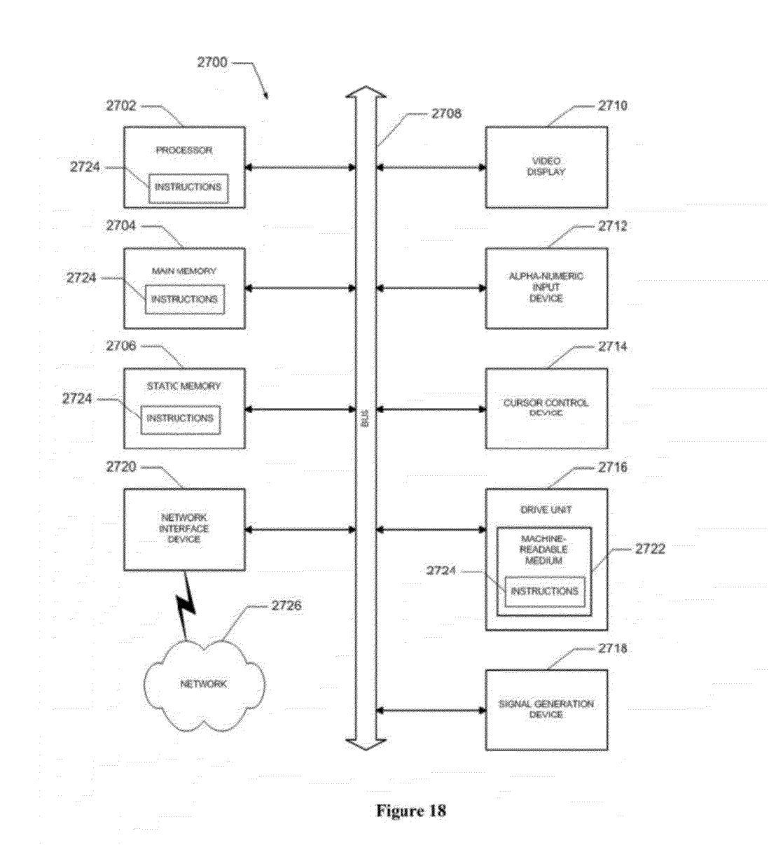

FIG. 18 shows a diagrammatic representation of machine in the example form of a computer system 2700 within which a set of instructions when executed may cause the machine to perform any one or more of the methodologies discussed herein. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” can also be taken to include any collection of machines that individually or jointly execute a set (or multiple sets) of instructions to perform any one or more of the methodologies discussed herein.

The example computer system 2700 includes a processor 2702 (e.g., a central processing unit (CPU), a graphics processing unit (CPU), or both), a main memory 2704 and a static memory 2706, which communicate with each other via a bus 2708. The computer system 2700 may further include a video display unit 2710 (e.g., a liquid crystal display (LCD) or a cathode ray tube (CRT)). The computer system 2700 also includes an input device 2712 (e.g., a keyboard), a cursor control device 2714 (e.g., a mouse), a disk drive unit 2716, a signal generation device 2718 (e.g., a speaker) and a network interface device 2720. The validation processor which is used for far wall estimation and IMT measurement is claimed to be running on a three tier architecture where the presentation layer can be a hand-held display device and the business logic and persistence layer can be the cloud. Such set-up is defined to be called as: AtheroCloud™.

The disk drive unit 2716 includes a machine-readable medium 2722 on which is stored one or more sets of instructions (e.g., software 2724) embodying any one or more of the methodologies or functions described herein. The instructions 2724 may also reside, completely or at least partially, within the main memory 2704, the static memory 2706, and/or within the processor 2702 during execution thereof by the computer system 2700. The main memory 2704 and the processor 2702 also may constitute machine-readable media. The instructions 2724 may further be transmitted or received over a network 2726 via the network interface device 2720. While the machine-readable medium 2722 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” can also be taken to include any non-transitory medium that is capable of storing, encoding or carrying a set of instructions for execution by the machine and that cause the machine to perform any one or more of the methodologies of the various embodiments, or that is capable of storing, encoding or carrying data structures utilized by or associated with such a set of instructions. The term “machine-readable medium” can accordingly be taken to include, but not be limited to, solid-state memories, optical media, and magnetic media.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

What is claimed is:

1. A computer-implemented method comprising:
collecting ultrasound image data for blood vessels;
using a data processor for alignment of raw grayscale ultrasound images with respect to a raw grayscale reference ultrasound image using binary alignment parameters;
using the data processor to generate the binary alignment parameters by combining a segmentation of grayscale ultrasound lumen images into binary lumen images and aligning the binary lumen images with a reference binary lumen image;
generating the segmentation of grayscale ultrasound lumen images by using a level set method and mathematical morphology in a Guidance Zone Region of Interest (ROI);
estimating the Guidance Zone using apriori knowledge and automated detection of a far adventitia border;
detecting the far adventitia border by using a multiresolution data processor;
using the data processor to generate a composite image of registered grayscale ultrasound images; and
using a validation data processor to estimate a highest brightness in a far wall of the blood vessels, the highest brightness of the far wall is automatically used to compute the intima-media thickness (IMT).

2. The method as claimed in claim 1 wherein the ultrasound image data comprises two-dimensional (2D) longitudinal B-mode or RF-mode ultrasound images and the ultrasound image data can be from different gain settings and from different machines.

3. The method as claimed in claim 1 where the composite image is generated by aligning the entire ultrasound image database with respect to a grayscale reference image using binary alignment parameters.

4. The method as claimed in claim 1 wherein generating the composite image includes alignment of grayscale ultrasound images with respect to the grayscale reference image, wherein the alignment parameters are generated by alignment of the binary lumen regional images.

5. The method as claimed in claim 1 wherein validation of the highest brightness of a far wall of the blood vessels uses a combination of the segmentation in conjunction with a rigid alignment or a non-rigid alignment, where the alignment parameters are computed using the regional lumen information.

6. The method as claimed in claim 1 wherein binary lumen images used for segmentation are computed using a combination of the level set method and the mathematical morphology in the guidance zone, which is created around the far adventitia borders computed automatically.

7. The method as claimed in claim 6 wherein the grayscale ultrasound images are edge enhanced using a special speed function to raise wall edges for ensuring that the level set method stops at these edges during level set evolution for binary lumen region segmentation, the special speed function being computed by subtracting every pixel in the image from the maximum value of the image and then multiplying the image by a function of the gradient of the original image, according to:
$$f ( ∇ u ) = \frac{1}{1 + \vert ∇ u \vert}$$
where, u indicates the image.

8. The method as claimed in claim 1 wherein binary lumen images used for segmentation are computed using a combination of the level set method and the mathematical morphology in the guidance zone, which is created around the far adventitia borders computed automatically, where as the far adventitia borders are estimated using automated multi-resolution strategy.

9. The method as claimed in claim 1 wherein alignment of grayscale ultrasound images uses either a rigid alignment or a non-rigid alignment of grayscale blood vessel ultrasound images for validating the highest brightness in the far wall of the blood vessels.

10. The method as claimed in claim 1 wherein the segmentation in conjunction with the registration for validation, of highest brightness in the far wall of the blood vessels can be used for finding the seed points in the far wall for estimation of Media Adventitia (MA) Borders useful for IMT measurement.

11. The method as claimed in claim 10 wherein the seed points on the highest brightness far wall of the blood vessels can be used to frame lumen-intima and media-adventitia borders of the far wall for IMT measurement, which is a representation of the cardiovascular risk due to Atherosclerosis in the blood vessels.

12. The method as claimed in claim 1 including using Atherosclerosis Monitoring (Atherometer™) based on tracing the IMT over time by using the same alignment of images of the same patient over time.

13. The method as claimed in claim 1 including using a mobile framework (AtheroMobile™) where the IMT can be computed in the far wall corresponding to the brightest wall as per a hypothesis.

14. The method as claimed in claim 1 wherein validation of highest brightness in blood vessels in ultrasound images uses the combination of segmentation and registration for validation of Hypothesis in four kinds of blood vessels: Carotid, Brachial, Aorta and Peripheral blood vessels.

15. The method as claimed in claim 1 wherein validation of highest brightness in blood vessels in ultrasound images uses the combination of segmentation and registration in a Cloud (AtheroCloud™) Computing framework wherein the blood vessel images can be downloaded from a Cloud and IMT can be computed in the Cloud corresponding to the highest brightness in the far wall, validated by the hypothesis.

16. The method as claimed in claim 1 wherein validation of highest brightness in blood vessels in ultrasound images uses the combination of segmentation and registration in a Cloud (AtheroCloud™) Computing framework wherein the blood vessel images can be downloaded from a Cloud and IMT can be computed in the Cloud corresponding to the highest brightness in the far wall, validated by the hypothesis, while the presentation is on the hand-held device such as tablet.

17. The method as claimed in claim 1 wherein validation of highest brightness in blood vessels in ultrasound images uses the combination of segmentation and registration for estimating the stenosis in the blood vessels in ultrasound imagery covering the application of carotid, brachial, aorta and peripheral.

18. The method as claimed in claim 15 including performing IMT measurement in an automated framework where LI and MA borders are automatically computed.

19. The method as claimed in claim 15 including performing IMT measurement for classification of plaque into symptomatic and asymptomatic plaques (Atheromatic™).

20. The method as claimed in claim 1 including combining data with independent systems including AtheroEdge, Atheromatic™, and AtheroRisk™ into a blood vessel analysis system such as VesselOmeasure™.
